(19) United States
(12) Patent Application Publication
(10) Pub. No.: US 2022/0036965 A1
(43) Pub. Date: Feb. 3, 2022

(54) METHOD

(71) Applicant: Technological University Dublin, Dublin (IE)

(72) Inventor: Andrew Knox, Dublin (IE)

(21) Appl. No.: 17/276,414

(22) PCT Filed: Sep. 16, 2019
(86) PCT No.: PCT/EP2019/074754

§ 371 Date: Mar. 15, 2021

(30) Foreign Application Priority Data
Sep. 14, 2018 (GB) 1815053.2

(51) Int. Cl. G16B 15/30; C07K 1/22; B01J 20/285; B01J 20/286; B01D 15/38

(52) U.S. Cl. CPC G16B 15/30; C07K 1/22; B01J 2220/52; B01J 20/286; B01D 15/3804; B01J 20/285

(57) ABSTRACT

There is provided a method of identifying a resin for isolating or enriching a protein of interest using affinity chromatography. The method comprises the steps of: i) providing the three-dimensional structure of the protein of interest; ii) determining and/or calculating one or more parameters of the protein of interest in its two- and/or three-dimensional form; iii) determining and/or calculating one or more parameters of one or more resin in their two- and/or three-dimensional form; and iv) selecting a resin expected to bind complementarily to the protein of interest based upon one or more of the parameters of the protein of interest.

The present invention relates to a method of identifying a resin for isolating or enriching a protein of interest using affinity chromatography, and to a method of identifying one or more preferred affinity ligands for isolating or enriching a protein of interest using affinity chromatography.

Proteins such as ovotransferrin, soy protein, and casein are high value and desirable to isolate and enrich from food or supplement production. Ovotransferrin from egg-white has been shown in numerous studies to harbour a broad range of health benefits (antibacterial, antitumorogenic, antiviral, etc.). Currently the only described production and purification processes requires either treatment of egg-white with alcohol, addition of heavy metals, treatment with organic solvents or precipitation with high-salt/organic acid concentrations which renders the remaining egg-white unusable.

Soybeans provide a good source of low-cost protein and have become an important world commodity because they are ubiquitous, have unique chemical composition, good nutritional value, versatile uses, and functional health benefits. Yet, less than about 5% of the soybean protein available is used for food due to the presence of anti-nutritional factors such as trypsin inhibitors, which prevent the uptake of nutrients from the food source. The most common method of reducing the activity of these inhibitors is to heat the soy protein which denatures/destroys not only the trypsin inhibitor proteins, but all other proteins in the matrix also and renders their functionality inactive. An alternative to heating the soy protein to destroy the trypsin inhibitor (TI) protein is to separate the trypsin inhibitor (TI) protein from the remainder of the soy protein. This technique has the advantages of avoiding heat, and can also provide isolated trypsin inhibitor (TI) protein (which itself can be a useful medicinal product).

The proteins in milk, which are mainly found as casein proteins or whey proteins, have gained increasing attention over the years. The reason for this increased interest lies in the diversity of milk proteins and because each protein has unique attributes to nutritional, biological, functional and food ingredient applications. The main protein component in milk is casein which is mainly found as micellar casein, formed by macromolecular casein aggregates.

Traditionally, treatment of milk generally consists of an initial extraction of casein, such as by precipitation of aggregated micellar casein, e.g. by enzymatic modification using rennet or by acid treatment, providing a precipitate of aggregated casein, a curd, and a liquid whey protein solution.

However, this treatment is disadvantageous because the enzymatic modification or the acidic treatment may cause the aggregated casein and/or part of the soluble proteins to be partly degraded and the proteins may lose some of the biological activity.

Furthermore, the precipitated casein may entrap some soluble proteins within the aggregate and thereby reducing the yield of soluble proteins or increase impurities in the aggregated casein precipitate.

Also, some proteins, such as ovotransferrin, are inherently sensitive towards different stress factors, like thermal stress and therefore prone to aggregation and denaturation.

Affinity chromatography is known and is employed to separate compounds and/or substances using the specific affinity between a substance fixed in the separation material (i.e. the resin) and the desired component in the mixture. The skilled person will understand that affinity chromatography methods include ion exchange chromatography.

Traditional methods have required a vast burden of time, cost and other resource spent on experimentation in order to find a resin that is suitable for isolating a protein of interest using affinity chromatography.

The skilled person would previously have had to purchase a wide variety of resins in order to find one that might exhibit a slight binding to the protein of interest. The skilled person would then have had to test each resin for binding with the protein of interest and manually interpret the results.

The skilled person could have required many iterations of this process in order to find a resin that binds to the protein of interest in a manner that is able to isolate or enrich the protein in a high enough yield to be commercially viable.

The present inventors have identified that small molecule ligands could provide a way to stabilise the protein in solution during the production process.

The present inventors have identified that the use of small molecule ligands could improve the yield and/or final activity of the protein preparations.

An aim of the invention is to provide alternative or enhanced methods of identifying binding ligands for the purification or enrichment of proteins of interest, for example from mixed protein sources.

According to a first aspect of the present invention, there is provided a method of identifying a resin for isolating or enriching a protein of interest using affinity chromatography comprising the steps of:
i) providing the three-dimensional structure of the protein of interest;
ii) determining and/or calculating one or more parameters of the protein of interest in its two- and/or three-dimensional form, such as:
   a) electrostatic potential;
   b) size;
   c) amino acid content and/or sequence;
   d) hydrophobicity/hydrophilicity;
   e) molecular weight;
   f) hydrogen bond donors and/or acceptors;
   g) π-stacking regions; and/or
   h) cation and/or π regions for cation-π interactions;
   iii) determining and/or calculating one or more parameters of one or more resin in their two- and/or three-dimensional form, such as:
   a) electrostatic potential;
   b) pore size;
   c) characteristics that will bind to amino acids of the protein of interest;
   d) hydrophobicity/hydrophilicity;
   e) average molecular weight;
   f) hydrogen bond donors and/or acceptors;
   g) π-stacking regions; and/or
   h) cation and/or π regions for cation-π interactions; and
iv) selecting a resin expected to bind complementarily to the protein of interest based upon one or more of the parameters of the protein of interest.

The method of the first aspect allows the skilled person to identify such a resin without the burdens associated with traditional methods.

By determining and/or calculating parameters of the protein of interest for the resin to interact with, the skilled person is taken straight to a resin that could isolate the protein of interest. The resin may be identified/designed computationally (in silico), allowing for rapid execution of the method.

A range of resins for affinity chromatography currently exist for the purification of a protein from a mixture containing other materials. In the present invention, it is preferred that the resin is a polysaccharide-based resin, for example a resin based on agarose, alginate, cellulose, chitin, starch, glycogen, callose, laminarin, chrysolaminarin, xylan, arabinoxylan, mannan, fucoidan, pectins and/or galactomannan. It may be understood that additives could be included in the resin to modify certain parameters of that resin, such as the electrostatic potential and/or pore size of the resin.

In a preferred embodiment the method is computer-implemented. In one embodiment the protein of interest is Ovotransferrin (PDB ID 1AIV).

Preferably the method includes providing the protein sequence of the protein of interest. Preferably this is performed prior to step (i).

In step (i), the three-dimensional structure of the protein may be:
a) retrieved from a database of the three-dimensional structures of proteins, such as the Protein Data Bank (PDB);
b) determined using homology modelling, for example using I-TASSER (Iterative Threading ASSEmbly Refinement) protein structure and function predictions available at https://zhanglab.ccmb.med.umich.edu/I-TASSER/;
c) determined using NMR techniques. Such techniques will apparent from the common general knowledge; and/or
d) determined using X-ray diffraction techniques. Such techniques will apparent from the common general knowledge.

The skilled person will understand that homology modelling can be achieved by identifying structural templates from the PDB by multiple threading approach LOMETS (Local Meta-Threading-Server), with full-length atomic models constructed by iterative template fragment assembly simulations. Function insights of the target are then derived by threading the 3D models through a protein function database such as BioLiP.

The three-dimensional protein surface electrostatics can be calculated, for example, using the DelPhi algorithm, and/or using the “DelPhiForce” method (L. Li, A. Chakravorty, E. Alexov. J. Comput. Chem. 2017, 38, 584-593; DOI: 10.1002/jcc.24715). DelPhiForce is a tool in the DelPhi package that calculates and visualizes the electrostatic forces in biomolecular systems. In parallel, the DelPhi algorithm for modeling electrostatic potential at user-defined positions has been enhanced to include triquadratic and tricubic interpolation methods. The DelPhiForce is further applied in the study of forces acting between partners of three protein-protein complexes. DelPhiForce is available for download from the DelPhi webpage at: http://compbio.clemson.edu/downloadDir/delphiforce.tar.gz

In one embodiment, the selection of a resin to bind complimentarily to the protein of interest is based upon two or more, such as three or more parameters, or four or more parameters. Basing the selection upon more than one parameter may allow for the resin to bind to the protein with greater specificity, for example over other proteins.

The parameter of the protein of interest may include the electrostatic potential of the protein, such as the two-dimensional or three-dimensional electrostatic potential. For example, the surface electrostatic potential may be calculated as a parameter of the protein of interest. In this case, the skilled person will understand in light of this disclosure that a negatively charged resin may be selected to isolate a positively charged protein, and vice versa. For example, alginate resins have a negatively charged surface due to exposed carboxylate moieties, and these resins typically have Ca²⁺ counterions. Such a negatively charged resin may find particular application in isolating proteins with a positive overall charge.

The parameter of the protein of interest may include the size of the protein, such as the two-dimensional or three-dimensional size of the protein. For example, the average or maximum diameter may be calculated as a parameter of the protein of interest. In this case, the skilled person will understand in light of this disclosure that a resin may be selected with a pore size that is larger than the size of the protein of interest, such as the average or maximum diameter of the protein of interest. The skilled person will also understand that proteins may tend to aggregate, to form aggregates, under certain conditions such as in a certain pH range and/or at certain concentrations. Therefore, the resin may be selected that has a pore size that is larger than the size of aggregates of the protein, for example under given conditions. For example the pore size may be up to about 40% or 50% larger than the protein of interest, or aggregates thereof.

The parameter of the protein of interest may include the amino acids of the protein, such as the amino acids in the two-dimensional or three-dimensional structure of the protein. For example, the amino acids on the surface of the three-dimensional structure of the protein of interest may be calculated. In this case, the skilled person will understand in light of this disclosure that a resin may be selected with characteristics that will bind to amino acids of the protein of interest, such as amino acids on the surface of the three-dimensional structure of the protein of interest.

The parameter of the protein of interest may include the hydrophobicity and/or the hydrophilicity of the protein, such as the hydrophobicity and/or the hydrophilicity of the two-dimensional or three-dimensional structure of the protein of interest. For example, the hydrophobicity and/or the hydrophilicity of the surface of the three-dimensional structure of the protein of interest may be calculated. In this case, the skilled person will understand in light of this disclosure that a hydrophobic resin may be selected to isolate a hydrophobic protein of interest, or a hydrophilic resin may be selected to isolate a hydrophilic protein of interest.

The parameter of the protein of interest may include the molecular weight of the protein.

The parameter of the protein of interest may include the hydrogen bond donors and/or acceptors of the protein, such as the hydrogen bond donors and/or acceptors of the two-dimensional or three-dimensional structure of the protein of interest. For example, the hydrogen bond donors and/or acceptors of the surface of the three-dimensional structure of the protein of interest may be calculated. In this case, the skilled person will understand in light of this disclosure that a resin high in hydrogen bond donors may be selected to isolate a protein of interest high in hydrogen bond acceptors, or vice versa, for example when the hydrogen bond donors and/or acceptors of the protein of interest are specifically on the surface of the three-dimensional structure of that protein.

The parameter of the protein of interest may include the π-stacking regions of the protein, such as the π-stacking regions of the two-dimensional or three-dimensional structure of the protein of interest. For example, π-stacking regions of the surface of the three-dimensional structure of the protein of interest may be calculated. In this case, the skilled person will understand in light of this disclosure that a resin with π-stacking regions may be selected to isolate a protein of interest with π-stacking regions, for example when the π-stacking regions of the protein of interest are on the surface of the three-dimensional structure of that protein.

The parameter of the protein of interest may include cation and/or π regions of the protein for cation-π interactions, such as the cation and/or π regions of the two-dimensional or three-dimensional structure of the protein of interest. For example, the cation and/or π regions of the surface of the three-dimensional structure of the protein of interest may be calculated. In this case, the skilled person will understand in light of this disclosure that a resin high cationic regions may be selected to isolate a protein of interest high in π regions, or vice versa, for example when the cation and/or π regions of the protein of interest are specifically on the surface of the three-dimensional structure of that protein.

In step (iii), one or more parameters of two or more resins, such as ten or more resins, or 100 or more resins may be calculated and/or determined. The larger the selection of proteins, the more likely it may be that a resin can be found with a favourable binding to the protein of interest.

A resin may be selected, based on the calculating one or more parameters of the protein of interest, using “Molecular Operating Environment”, distributed by Chemical Computing Group.

According to a second aspect of the present invention, there is provided a method of identifying one or more preferred ligands for isolating a protein of interest using affinity chromatography, the method comprising the steps of:
i) providing the three-dimensional structure of the protein of interest and creating a model of a receptor-based pharmacophore of the protein of interest using the three-dimensional structure of the protein of interest, and determining and/or calculating one or more parameters of the model of the receptor-based pharmacophore in its two- and/or three-dimensional form, such as:
   a) electrostatic potential;
   b) size;
   c) amino acid content and/or sequence;
   d) hydrophobicity/hydrophilicity;
   e) molecular weight;
   f) hydrogen bond donors and/or acceptors;
   g) π-stacking regions; and/or
   h) cation and/or π regions for cation-π interactions;
ii) providing a database of molecules;
iii) selecting, from the database, molecules that include primary amines and/or carboxylic acid moieties;
iv) screening the selected molecules against the model of the receptor-based pharmacophore to find one or more molecules expected to bind complementarily to the protein of interest based upon one or more of the parameters of the model of the receptor-based pharmacophore of the protein of interest;
v) selecting, as one or more potential ligands, the one or more molecules expected to bind complimentarily to the protein of interest;
vi) calculating the binding affinity of the one or more potential ligands with the protein of interest using a docking algorithm; and
vii) selecting, as one or more preferred ligands, one or more potential ligands with the highest binding affinity.

The binding affinity calculated may be the predicted binding affinity.

The application of this methodology to the design of both ligand diversity sets for protein enrichment and also for design of specific affinity ligands is both new and surprisingly effective.

The preferred ligands may be identified/designed computationally (in silico), allowing for rapid execution of the method. In a preferred embodiment the method is computer-implemented. In one embodiment the protein of interest is ovotransferrin (PDB ID 1AIV).

In one embodiment, the methods of the first and the second methods may be used in conjunction, to identify both a suitable ligand and a suitable resin, for enhanced results.

Docking studies involve the rotation and translation of a compound across the surface of the pharmacophore of protein of interest. This is typically performed by computers due to the large amount of calculation required. Even with computers, docking studies are very resource-intensive, especially with respect to computer resources such as processing power and time.

The initial screening of the selected molecules, as described in step (iii), allows for the number of molecules to be subjected to docking studies can be greatly reduced. Therefore, the burden on computer resource can be greatly reduced by having an initial screening step before the docking studies are commenced.

In one embodiment, the method of the second aspect further includes the steps of:
viii) obtaining one or more preferred ligands and determining their ability to bind the protein of interest, optionally using surface plasmon resonance;
ix) immobilising positive binding ligands on a bead to further determine binding ability in a binding assay, optionally using sodium dodecyl sulfate polyacrylamide gel electrophoresis (SDS page); and optionally further comprising the step of:
x) in silico optimising the binding affinity of one or more positive binding ligands and validating the binding affinity with a further binding assay.

The skilled person will understand that preferred binding ligands are considered to be positive binding ligands.

The positive binding ligands may be anchored to a resin bead, such as those described herein, for use in using affinity chromatography purification, enrichment or isolation of the protein of interest.

In step (i), the three-dimensional structure of the protein may be:
a) retrieved from a database of the three-dimensional structures of proteins, such as the Protein Data Bank (PDB);
b) determined using homology modelling, for example using I-TASSER (Iterative Threading ASSEmbly Refinement) protein structure and function predictions available at https://zhanglab.ccmb.med.umich.edu/I-TASSER/;
c) determined using NMR techniques. Such techniques will apparent from the common general knowledge; and/or
d) determined using X-ray diffraction techniques. Such techniques will apparent from the common general knowledge.

Preferably the model of the receptor based pharmacophore of the protein of interest is a consensus of more than one model. Preferably the model, such as the consensus model, of the receptor based pharmacophore of the protein of interest is created using input from one or two or three of FTMAP, Autoligand, RaptorX and BSPRED. In one embodiment an input from FTMAP, Autoligand, RaptorX and BSPRED is used.

FTMAP is available at http://ftmap.bu.edu. Autoligand is available at http://autodock.scripps.edu/resources/autoligand. RaptorX is available at http://raptorx.uchicago.edu/BindingSite/. BSPRED is available at https://zhanglab.ccmb.med.umich.edu/BSpred/.

The database of molecules described in step (ii) could be Enamine, available at https://enamine.net/; however, other databases are available and could be alternatively or additionally used. In one embodiment the database is a database of organic molecules, for example organic molecules having a molecular weight below 1000 g/mol.

In one embodiment, molecules that include primary amines and carboxylic acid moieties are selected from the database. In one embodiment, molecules that include primary amines or carboxylic acid moieties are selected from the database.

It has been identified that the selection of molecules that contain primary amines and/or carboxylic acids is particularly beneficial. This requirement enables the selected molecules to covalently bond to a resin.

In one embodiment, the selected molecules may be screened and selected based on one or more parameters of the protein of interest, using “Molecular Operating Environment”, distributed by Chemical Computing Group.

In one embodiment, screening step (iv) may include determining and/or calculating one or more parameters of the selected molecules in their two- and/or three-dimensional form, such as:
a) electrostatic potential;
b) pore size;
c) characteristics that will bind to amino acids of the protein of interest;
d) hydrophobicity/hydrophilicity;
e) average molecular weight;
f) hydrogen bond donors and/or acceptors;
g) π-stacking regions; and/or
h) cation and/or π regions for cation-π interactions.

The descriptions of complementary parameters given in relation to the first aspect apply equally in relation to the second aspect, save that the resin is replaced by the selected molecules.

In one embodiment of step (v), 20 or more molecules, such as 50 or more molecules are selected as potential ligands. In one embodiment, 100 or fewer, or 200 or fewer molecules are selected. In one embodiment, the selected molecules are those with the strongest binding interaction with the model of the receptor-based pharmacophore.

In one embodiment of step (vii), 20 or fewer, such as 10 or fewer, or 5 or fewer molecules are selected as preferred ligands, or one molecule is selected as a preferred ligand. Whilst a number of docking algorithms are available, for example, BSP-SLIM may be used. BSP-SLIM is available at https://zhanglab.ccmb.med.umich.edu/BSP-SLIM/.

Preferably the docking algorithm is performed using one or two or three of FTMAP, Autoligand, RaptorX and BSPRED. In one embodiment the docking algorithm is performed using FTMAP, Autoligand, RaptorX and BSPRED.

The one or more ligands with the highest binding affinity may be determined using a number of scoring functions, as will be immediately apparent to the skilled person. The scoring function used may be determined by the software used to perform the docking study. In one embodiment, the scoring function is an empirical scoring function that is, for example, based upon the number of hydrogen bond donor-acceptor interactions generated between the pharmacophore and the ligand.

In the optional in silico optimisation of the binding affinity, the experimental binding affinity of one or more ligands to the protein of interest may be correlated with the parameters of the protein in order to potentially determine ligands with yet higher binding affinity to the protein of interest.

The skilled person will understand that databases, such as Enamine, may be provided initially as a “flat” file. In order to exploit a database to its full capability, it may be necessary to convert in to three-dimensional models. This is performed by generating conformers, which can be screened against. Software such as Corina (https://www.mn-am.com/products/corina) can be used to generate such conformers.

The methods of the present invention may be used in conjunction with a software application, for example, executable on a mobile test reader (i.e., a computing or processing device). This is intended to be construed broadly, and to cover personal and mobile computing devices as well as other intelligent devices comprising a processing means. The software application may be accessible by a user on any appropriate computing or processing device such as a mobile phone, wearable, watch, tablet, laptop or other personal electronic and/or computing device (such as a digital signal processor, a microcontroller, and an implementation in read only memory (ROM) or electronically erasable programmable read only memory (EEPROM), as non-limiting examples). The software application may be an assembly program.

The software application, including any saved data generated by the software application, may be stored locally on the mobile test reader, or remotely from the mobile test reader (e.g., in a cloud or other storage means, online or otherwise), and may be accessed via the internet or otherwise. The software application may be provided on a computer readable medium, which may be a physical computer readable medium, such as a disc or a memory device, or may be embodied as a transient signal. Such a transient signal may be a network download, including an internet download.

According to another aspect of the invention, there is provided a method of isolating or enriching a protein of interest from a protein mixture,
wherein the method comprises isolating, purifying or enriching the protein of interest using affinity chromatography with a resin that has been selected or made according to the first and/or second aspect of the invention.

The protein mixture may comprise raw material, industrial side-streams, or waste material. The protein mixture may comprise plant material or animal product, such as meat, milk or egg. The protein of interest may be any protein, or may comprise any one of ovotransferrin, soy protein, casein or whey.

### EXAMPLES

This methodology enables the selection of a highly diverse set of molecules that are amenable to covalent immobilization ‘on bead’ for use in affinity chromatography.

The first use of this diversity set is to enable protein enrichment from complex protein matrices (e.g raw material, industrial side-streams, waste material) and to guide further optimisation in a similar fashion to the use of ‘on-bead’ combinatorial libraries for use in protein target binding in the pharmaceutical industry.

The process can be run in high throughput using miniaturized columns on a 96-well plate to allow probing of protein capture when integrated with high-performance liquid chromatography or LC-MS/MS.

Once a protein of interest is captured via a specific set of beads, the binding interactions can be investigated computationally to permit protein purification in a second step.

The specific design of one or a series of small molecule ligand(s) or selection of analogs capable of binding to the surface of the protein of interest is also described. In this way the user may be able to optimise again in a high-throughput fashion, the precise ligand and bead preparation required to produce optimal protein purification conditions.

As a proof of concept, a specific ligand has been computationally identified that enables the purification of a high-value egg-white protein, Ovotransferrin, which has been shown in numerous studies to harbour a broad range of health benefits (antibacterial, antitumorogenic, antiviral, etc.). Currently, the only described production and purification processes for ovotransferrin require treatment of egg-white with alcohol, addition of heavy metals, treatment with organic solvents or precipitation with high-salt/organic acid concentrations, which renders the remaining egg-white unusable.

Ovotransferrin is inherently sensitive towards different stress factors, like thermal stress and therefore prone to aggregation and denaturation. Small molecule ligands could also provide a way to stabilise the protein in solution during the production process and to improve the yield and final activity of the protein preparations.

Importantly, using the method of the invention, we have found a ligand that binds to Ovotransferrin and enables its separation from other egg-white components when immobilised in a bead.

To achieve highest possible potential of proteins and to explore or exploit the potentially functional and bioactive properties of proteins (e.g. proteins in milk, eggs, soybean etc.), it is important to isolate native proteins from complex matrices by procedures that avoid possible denaturing conditions (such as, high salt conditions, high or low pH conditions, heat or protease treatment/exposure). We outline next two examples of issues observed in the commercial isolation of proteins from soybean and milk respectively where our technology would produce benefits.

Soybean; Soybeans provide a good source of low-cost protein and have become an important world commodity because they are ubiquitous, have unique chemical composition, good nutritional value, versatile uses, and functional health benefits. Yet, less than about 5% of the soybean protein available is used for food due to the presence of anti-nutritional factors such as trypsin inhibitors, which prevent the uptake of nutrients from the food source. The most common method of reducing the activity of these inhibitors is to heat the soy protein which denatures/destroys not only the trypsin inhibitor proteins, but all other proteins in the matrix also and renders their functionality inactive. An alternative to heating the soy protein to destroy the trypsin inhibitor (TI) protein is to separate the trypsin inhibitor (TI) protein from the remainder of the soy protein. This technique has the advantages of avoiding heat, and can also provide isolated trypsin inhibitor (TI) protein (which itself can be a useful medicinal product).

Milk; The proteins in milk, which are mainly found as casein proteins or whey proteins, have gained increasing attention over the years. The reason for this increased interest lies in the diversity of milk proteins and because each protein has unique attributes to nutritional, biological, functional and food ingredient applications. The main protein component in milk is casein which is mainly found as micellar casein, formed by macromolecular casein aggregates. Traditionally, treatment of milk generally consists of an initial extraction of casein, such as by precipitation of aggregated micellar casein, e.g. by enzymatic modification using rennet or by acid treatment, providing a precipitate of aggregated casein, a curd, and a liquid whey protein solution. However, this treatment is disadvantageous because the enzymatic modification or the acidic treatment may cause the aggregated casein and/or part of the soluble proteins to be partly degraded and the proteins may lose some of the biological activity. Furthermore, the precipitated casein may entrap some soluble proteins within the aggregate and thereby reducing the yield of soluble proteins or increase impurities in the aggregated casein precipitate.

1. A method of identifying a resin for isolating or enriching a protein of interest using affinity chromatography, comprising the steps of:
i) providing the three-dimensional structure of the protein of interest;
ii) determining and/or calculating one or more parameters of the protein of interest in its two- and/or three-dimensional form;
iii) determining and/or calculating one or more parameters of one or more resin in their two- and/or three-dimensional form; and
iv) selecting a resin expected to bind complementarily to the protein of interest based upon one or more of the parameters of the protein of interest.

2. The method of claim 1, wherein the one or more parameters of the protein of interest comprise one or more of:
a) electrostatic potential;
b) size;
c) amino acid content and/or sequence;
d) hydrophobicity/hydrophilicity;
e) molecular weight;
f) hydrogen bond donors and/or acceptors;
g) π-stacking regions; and/or
h) cation and/or π regions for cation-π interactions.

3. The method of claim 1, wherein the one or more parameters of one or more resins comprise one or more of:
a) electrostatic potential;
b) pore size;
c) characteristics that will bind to amino acids of the protein of interest;
d) hydrophobicity/hydrophilicity;
e) average molecular weight;
f) hydrogen bond donors and/or acceptors;
g) π-stacking regions; and/or
h) cation and/or π regions for cation-π interactions.

4. The method of claim 1, wherein the resin is or comprises a polysaccharide-based resin, optionally, a resin based on agarose, alginate, cellulose, chitin, starch, glycogen, callose, laminarin, chrysolaminarin, xylan, arabinoxylan, mannan, fucoidan, pectins and/or galactomannan

5. The method of claim 1, further comprising providing the protein sequence of the protein of interest, optionally, prior to step (i).

6. The method of claim 1, wherein the three-dimensional structure of the protein is:
a) retrieved from a database of the three-dimensional structures of proteins, such as the Protein Data Bank (PDB);
b) determined using homology modelling;
c) determined using NMR techniques; and/or
d) determined using X-ray diffraction techniques.

7. The method of claim 1, wherein the resin is selected based upon two or more, three or more, or four or more parameters of the protein.

8. A method of identifying one or more preferred ligands for isolating a protein of interest using affinity chromatography, the method comprising the steps of:
i) providing the three-dimensional structure of the protein of interest and creating a model of a receptor-based pharmacophore of the protein of interest using the three-dimensional structure of the protein of interest, and determining and/or calculating one or more parameters of the model of the receptor-based pharmacophore in its two- and/or three-dimensional form;
ii) providing a database of molecules;
iii) selecting, from the database, molecules that include primary amines and/or carboxylic acid moieties;
iv) screening the selected molecules against the model of the receptor-based pharmacophore to find one or more molecules expected to bind complementarily to the protein of interest based upon one or more of the parameters of the model of the receptor-based pharmacophore of the protein of interest;
v) selecting, as one or more potential ligands, the one or more molecules expected to bind complimentarily to the protein of interest;
vi) calculating the binding affinity of the one or more potential ligands with the protein of interest using a docking algorithm; and
vii) selecting, as one or more preferred ligands, one or more potential ligands with the highest binding affinity.

9. The method of claim 8, wherein the binding affinity calculated is a predicted binding affinity.

10. The method of claim 8, wherein the one or more parameters of the model of the receptor-based pharmacophore comprise one or more of:
a) electrostatic potential;
b) size;
c) amino acid content and/or sequence;
d) hydrophobicity/hydrophilicity;
e) molecular weight;
f) hydrogen bond donors and/or acceptors;
g) π-stacking regions; and/or
h) cation and/or π regions for cation-π interactions.

11. The method of claim 8, further including the steps of:
viii) obtaining one or more preferred ligands and determining their ability to bind the protein of interest, optionally using surface plasmon resonance;
ix) immobilising positive binding ligands on a bead to further determine binding ability in a binding assay, optionally using sodium dodecyl sulfate polyacrylamide gel electrophoresis (SDS page); and, optionally, further comprising the step of:
x) in silico optimising the binding affinity of one or more positive binding ligands and validating the binding affinity with a further binding assay.

12. (canceled)

13. The method of claim 8, wherein the three-dimensional structure of the protein is:
a) retrieved from a database of the three-dimensional structures of proteins, such as the Protein Data Bank (PDB);
b) determined using homology modelling;
c) determined using NMR techniques; and/or
d) determined using X-ray diffraction techniques.

14. The method of claim 8, wherein molecules that include primary amines and/or carboxylic acid moieties are selected from the database.

15. The method of claim 8, wherein the screening step (iv) includes determining and/or calculating one or more parameters of the selected molecules in their two- and/or three-dimensional form; and, optionally, wherein the one or more parameters of the selected molecules comprise one or more of:
a) electrostatic potential;
b) pore size;
c) characteristics that will bind to amino acids of the protein of interest;
d) hydrophobicity/hydrophilicity;
e) average molecular weight;
f) hydrogen bond donors and/or acceptors;
g) π-stacking regions; and/or
h) cation and/or π regions for cation-π interactions.

16. (canceled)

17. The method of claim 8, wherein in step (v), 20 or more molecules, or 50 or more molecules are selected as potential ligands; and/or wherein in step (vii), 20 or fewer, 10 or fewer, or 5 or fewer molecules are selected as preferred ligands, or one molecule is selected as a preferred ligand.

18. (canceled)

19. The method of claim 1, wherein the method is computer-implemented.

20. A method of isolating or enriching a protein of interest from a protein mixture, wherein the method comprises isolating, purifying or enriching the protein of interest using affinity chromatography with a resin that has been selected or made according to claim 1.

21. The method of claim 17, wherein the protein mixture comprises raw material, industrial side-streams, or waste material; and/or wherein the protein mixture comprises plant material or animal product; and/or wherein the protein of interest is or comprises any one of ovotransferrin, soy protein, casein or whey.

22. (canceled)

23. (canceled)

24. A method of isolating or enriching a protein of interest from a protein mixture,
wherein the method comprises isolating, purifying or enriching the protein of interest using affinity chromatography with a resin that has been selected or made according to claim 8.

25. The method of claim 19, wherein the protein mixture comprises raw material, industrial side-streams, or waste material; and/or wherein the protein mixture comprises plant material or animal product; and/or wherein the protein of interest is or comprises any one of ovotransferrin, soy protein, casein or whey.
